(19) United States
(12) Patent Application Publication
(10) Pub. No.: US 2020/0315960 A1
(43) Pub. Date: Oct. 8, 2020

(54) DRUG DELIVERY DEVICE AND METHOD

(71) Applicant: BIORINGS LLC, New York, NY (US)

(72) Inventors: Sidney Lerner, New York, NY (US); Mukul Singh, Teaneck, NJ (US); Bruce Frank, Doylestown, PA (US); Onajite Okoh, Yeadon, PA (US)

(21) Appl. No.: 16/374,089

(22) Filed: Apr. 3, 2019

(51) Int. Cl. A61K 9/00; A61F 6/06

(52) U.S. Cl. CPC A61K 9/0036; A61M 31/002; A61F 6/06

(57) ABSTRACT

A biocompatible intravaginal device adapted for the delivery of therapeutic active ingredients (agents) is provided. The device is constructed to be insertable and remain in the vagina for up to a month (28 to 31 days) or more. The device is loaded with the active agents prior to insertion and constructed to continuously release a therapeutically effective amount of the agents during the duration of up to a one month insertion. Preferred agents include contraceptive and anti-infection agents. It is preferred that the agents are non-hormonal.

### BACKGROUND OF THE INVENTION

The invention relates generally to a drug delivery device and more particularly to an insertable device that will elute a controlled, effective amount of a therapeutic substance over a defined time. The invention also relates to a device for the long-term continued intravaginal delivery of spermiostatic, spermicidal, and anti-infectious agents, and methods for contraception and the prevention and treatment of infection using such a device.

Currently, intravaginal barrier and intrauterine contraceptive devices, with or without hormones, are available to inhibit ovulation and to prevent sperm migration into the cervix and fertilization. See, e.g., Roy, “Status of Research and Development of Vaginal Contraceptive Rings as Fertility Control Method in the Female,” Research Frontiers in Fertility Regulation, Family Health Network International Bulletin 2(4):1-10 (2000), the contents of which are incorporated herein by reference.

U.S. Pat. No. 8,268,343, titled Non-hormonal Vaginal Contraceptive, the contents of which are incorporated herein by reference, describes a biodegradable contraceptive device containing therapeutic ingredients. However, the '343 patent does not adequately describe how to design the device to control the rate of release and/or to have fully satisfactory, continued, sustained release.

Cellulose-based vehicles consisting of hydroxyethyl cellulose and hydroxyethyl methyl cellulose, or mixtures thereof, or optionally a cosmetic-type ingredient selected from the group consisting of water, ethyl alcohol, isopropyl alcohol, glycerin, glycerol, propylene glycol, and sorbitol, have also been used in delivery systems. Typical forms of delivery systems used vaginally include creams, lotions, gels, foams, sponges, suppositories, and films. However, controlled release rates remain an issue for long-term sustained release methods.

The success of a contraceptive depends not only upon the efficacy of the contraceptive method, but also upon the user's preference, reversibility, convenience, and compliance. Besides pregnancy, sexual relations can also transmit infection. It is thus beneficial that the design of contraceptive devices should also consider the option of protecting women against transmission of sexually transmitted diseases (STDs) as well as against pregnancy. Hormone-based contraceptives have long been identified as posing an adverse metabolic risk, and are, in fact, contraindicated for certain individuals with a variety of cardiovascular conditions. Therefore, it can be preferred that contraceptive devices are free of toxic compounds and hormones. However, a controlled release delivery vehicle of bioactive agents for contraception over extended periods has thus far not been adequately developed.

Therefore, there remains a need to develop a non-hormonal, biocompatible, cost-effective, and convenient long term device to prevent pregnancy and/or infection. The present invention is directed to overcoming these and other deficiencies in the art.

### SUMMARY OF THE INVENTION

Generally speaking, in accordance with the invention, a biocompatible intravaginal device adapted for the delivery of a controlled release of therapeutic ingredients (agents) is provided. The device is constructed to be insertable (implantable) and remain in the vagina for up to a month (31 days) or more. The device is charged with the agents prior to insertion and constructed to continuously release a therapeutically effective amount of the agents during the duration of insertion, whether 7, 21 or 31 days or more. Moreover, initial bursts of high levels of the active ingredients should be prevented. Preferred agents include contraceptive and anti-infection agents. It is preferred that the agents are non-hormonal. Preferred durations of release include two weeks, three weeks, four weeks or longer.

The device should be a flexible structure, preferably sufficiently open to permit the flow of vaginal fluids past the device. Ring shapes are preferred. The device can be formed from an elongated portion formed into a ring shape defining a central portion. The central portion of the ring can be partially obstructed, but should have openings so as not to be fully obstructed, so as not to completely cap the cervix. Preferred polymers include ethylene-vinyl acetate copolymers (EVA), preferably about 32-38% vinyl acetate by weight, more preferably about 35 wt %. In one preferred embodiment of the invention, the impregnated polymer matrix is partially sheathed to restrict the initial release of the active agents, which can provide a reservoir for the agents as insertion time progresses and limit the initial burst of eluted ingredients when the device is initially inserted.

Preferred contraceptive agents are included to: 1) reduce sperm motility an effective amount to prevent fertilization; 2) increase the viscosity of cervical mucus to effectively impede sperm motility to prevent fertilization; and 3) sustain a pH of approximately 4-6, preferably 5.0 or lower, in the vaginal cavity to augment the total spermiostatic effect involving reduced sperm mobility and increased viscosity.

In addition, a contraceptive method should allow women to use the method themselves in conjunction with normal management of their menstrual cycle as a tampon exchange month after month, thus enhancing the quality of life. Therefore, preferred methods of use include replacement of the device each month at the conclusion of the menstrual cycle.

Still other objects of the invention will in part be obvious and will, in part be apparent from the specification and drawings. The invention accordingly comprises the article of manufacture, method of using the article, and the method of making the article, which will be more fully exemplified in the articles and methods hereinafter described, and the scope of the invention will be indicating the claims.

### BRIEF DESCRIPTION OF THE DRAWINGS

For a fuller understanding of the invention, reference is had to the following description, taken in connection with the accompanying drawings, in which:

FIG. 1 is a perspective view of an insertable ring in accordance with an embodiment of the invention;

FIG. 2 is a cross sectional view of the ring of FIG. 1;

FIG. 3 is a perspective view of a partially sheathed insertable ring in accordance with an embodiment of the invention;

FIG. 4 is a cross sectional view of the sheathed portion of the ring of FIG. 3; and

FIG. 5 is a cross sectional view of the un-sheathed portion of the ring of FIG. 3.

As used herein, identical reference numerals will indicate similar structures. The drawings, which are not necessarily drawn to scale, are for purposes of illustration only and are not intended to be interpreted as limiting the scope of the invention.

### DETAILED DESCRIPTION OF THE PREFERRED EMBODIMENTS

The invention relates to a device that can be inserted by a user or optionally another, well suited to be a non-hormonal, biocompatible intravaginal device for delivery of spermiostatic and/or spermicidal, and/or anti-infective agents. This device can be a flexible structure, preferably with a partially or fully open center. It should be impregnated with an effective amount (concentration) of biocompatible spermiostatic agents and/or spermicidal agents, and/or anti-infective agents to provide a contraceptive or anti-infective effect. Ring shapes are preferred. The device should be shaped so as not to completely cap the cervix and to permit the flow of vaginal fluids past the device.

Preferred embodiments of the invention include hormonal or more preferably, non-hormonal, biocompatible intravaginal devices for delivery of therapeutic agents. The device should retain sufficient agent to permit contraceptively and/or anti-infectively effective elution of the agent for at least one week. Preferable devices can elute effective amounts of the agent for three weeks. The most preferred devices can continuously elute effective amounts of the agent for over 28 days, even 31 days or more. It is therefore helpful to design the device to prevent too much of the impregnated agent(s) from eluting during the first one, two or three weeks. It is also preferred to prevent initial bursts of undesirably high levels of the impregnated active ingredients.

As used herein, the term non-hormonal includes the use of materials in the device of embodiments of the invention that do not include estrogen, progesterone, other steroids, or derivatives thereof, which are systemic in action. In contrast, the materials suitable for the present invention can be non-hormonal, non-steroidal, and act locally at the site of insertion. The basic design of the delivery vehicle of preferred embodiments of the invention is a biocompatible polymer, which may be either natural and/or synthetic.

An important objective of the design is to facilitate the sustained release of effective amounts of the impregnated agents for up to a 31-day period, providing predictable, controlled release of the impregnated drugs. Initial burst releases should be controlled. Devices in accordance with the invention should minimize mechanical and frictional irritation to the surrounding tissue. Soft and compliant materials are preferred. Devices in accordance with preferred embodiments of the invention can provide a far more user-friendly delivery vehicle than other carriers.

The invention also relates to methods of contraception. This method involves introducing a device according the present invention into the vagina of a female mammal. The invention also relates to a method of preventing infection in mammals. The invention also relates to a method of treating vaginal infections in mammals. This method involves introducing the device of the present invention into the vagina of a female mammal.

As used herein, spermiostatic herein refers to the ability to retard sperm motility in an effective amount so as to prevent pregnancy. Spermicidal refers to the ability to kill sufficient sperm to prevent pregnancy. Sperm may be rendered ineffective physiologically when sperm have been irreversibly immobilized, even if not killed. See, Olmsted et al., “The Rate at Which Human Sperm Are Immobilized and Killed by Mild Acidity,” Fertility And Sterility 73(4) 687-693 (2000), which is hereby incorporated by reference in its entirety.

A spermiostatic/spermicidal aspect of the present invention can be provided by a one to three-pronged attack. Specific agents (active ingredients) can be included to: 1) reduce sperm motility an effective amount to prevent fertilization; 2) increase the viscosity of cervical mucus to effectively impede sperm motility to prevent fertilization; and/or 3) sustain a pH of approximately 5.0 or lower, in the vaginal cavity to augment the total spermiostatic effect involving reduced mobility and increased viscosity. Spermiostatic/spermicidal agents suitable for the present invention include, but are not limited to, magnesium chloride, calcium chloride, ferrous sulfate, copper sulfate, ferrous gluconate, and mixtures thereof. Ferrous gluconate and ferrous sulfate are preferred.

The use of these metallic salts as spermiostatic agents, and concentrations of these materials that are effective for spermiostatic efficacy are known to those skilled in the art (see, for example, U.S. Pat. No. 4,959,216 to Daunter; and Shoham et al., “Influence of Different Copper Wires on Human Sperm Penetration Into Bovine Cervical Mucus,” In Vitro Contraception 36(3):327-34 (1987), which are hereby incorporated by reference in their entirety).

The secretory cells of the mucosa of the cervix produce a secretion called cervical mucus, a mixture of water, glycoprotein, serum-type proteins, lipids, enzymes, and inorganic salts. Females of reproductive age commonly secrete 20-60 ml of cervical mucus per day. Cervical mucus is more receptive to sperm at or near the time of ovulation because it is less viscous. In addition, cervical mucus becomes more alkaline, with a pH of about 7.5-8.5, in the presence of semen, which can enhance chances for fertilization. After ovulation, whether or not sexual relations have occurred, the mucus becomes thicker and forms a cervical plug that is physically impenetrable to sperm. Thereafter, the cycle repeats, with the mucus becoming less viscous as ovulation approaches and thicker afterwards.

Therefore, if the viscosity of the cervical mucus is increased sufficiently during the period of the cycle when it is less viscous, sperm motility can be impeded and fertilization prevented. One preferred agent suitable for increasing the viscosity of the cervical mucous that can be eluted from a device of the present invention is ascorbic acid (L-ascorbic acid). It has been shown that L-ascorbic acid, more commonly known as Vitamin C, is successful in triggering the above-described viscosity increasing chain of events. Ascorbic acid can act as a reducing agent on the mucopolysaccharides of the cervical mucus. It transfers electrons to the mucopolysaccharides, causing the cervical mucus to change conformation. The open cellular structure that the mucus cells originally have is subsequently closed, thus causing an increase in viscosity. This increased viscosity results in inhibited sperm motility. The increase in the viscosity of the cervical mucus induced by the release of L-ascorbic acid from the delivery device of the invention serves as an effective line of resistance to help prevent the sperm from reaching the ovum.

The optimum pH value for sperm migration and sperm survival in the cervical mucus is believed to be slightly alkaline, between 7.5 and 8.5. Acidic mucus has been determined to immobilize sperm in the vagina, thus aiding contraception (WHO Laboratory Manual for the Examination of Human Semen and Sperm-cervical Mucus Interaction, Ch. 5:51-59 (1999), which is hereby incorporated by reference in its entirety). It has been determined that the immobilization of sperm that occurs in the vagina at a pH of or about 5.0 or lower can help prevent fertilization by creating an environment wherein the sperm are irreversibly immobilized (Olmsted et al., “The Rate at Which Human Sperm Are Immobilized and Killed by Mild Acidity,” Fertility And Sterility 73(4):687-693 (2000), which is hereby incorporated by reference in its entirety).

A device of an embodiment of the invention can function to sustain the vaginal pH at or about 5.0 or lower in two ways. First, buffered acid sources, such as poly-amino and polycarboxylic acid mixtures (e.g., ampholines), with a pH range of 4-6, can be incorporated into the device. As these are released, they help maintain the vaginal pH in the acidic range (at or about pH 5.0 or lower), even in the presence of semen.

One of the prime advantages of this unique three-pronged approach to contraception provided by the present invention is that the combination provides for greater efficacy and dependability than other contraceptive measures which incorporate any one, or even two, of these approaches in a single contraceptive. Furthermore, because the multiple prongs contribute simultaneously to the immobilization and death of sperm, relatively low concentrations of spermiostatic/spermicidal agents are needed. Furthermore, the non-hormonal, non-systemic, and biodegradable nature of the present invention provides a method of contraception that can be used regularly and long-term without negative repercussions to users' health. Furthermore, by controlling the initial and long term rate at which the active ingredients are released, continuous effective amounts can be released for two weeks, four weeks, 31 days or longer.

Another aspect of the present invention includes a method of preventing infection in mammals including, but not limited to, humans, by introducing a device of the invention in the vagina of a female mammal. This additional advantage can be accomplished by incorporating anti-infectious agents into the device, with or without the spermiostatic agents. Anti-infective agents suitable for the present invention include anti-viral agents, anti-fungal agents, antibiotics, and mixtures thereof. This includes prophylactic treatment against sexually transmitted diseases (“STDs”) such as HIV, particularly for those in high risk populations. Depending on the intended application of the device, the anti-infective agents of the present invention can be used with or without the spermiostatic and/or spermicidal agents described above.

Examples of inti-infective active ingredients that can be incorporated into a device in accordance with embodiments of the invention include:

Bacterial infections: clindamycin (Cleocin) and metronidazole (MetroGel-Vaginal).
Viral infections: Pre-exposure prophylaxis, or PrEP, (brand name Truvada) contains two medicines (tenofovir and emtricitabine).
Yeast Infections: Yeast infections can often be treated with miconazole (Monistat 1), clotrimazole (Gyne-Lotrimin), butoconazole (Femstat 3), or tioconazole (Vagistat-1)

The effects of various concentrations of magnesium chloride, calcium chloride, ferrous sulfate, copper sulfate, and ferrous gluconate on the motility of human sperm were studied. Calcium chloride (CaCl₂)) and magnesium chloride (MgCl₂) were shown to be spermiostatic at concentrations of 25 mM and 35 mM, respectively. Ferrous sulfate (FeSO₄) completely arrested the motility of human sperm at a concentration of 10 mM. Copper sulfate and ferrous gluconate were spermiostatic at concentrations of 6.25 mM and 12.5 mM, respectively. 25 mM solutions of copper sulfate and ferrous gluconate showed 93.3% and 97.4% immobilization of sperm, respectively. 37.5 mM solutions of both reagents completely immobilized all the sperm. However, in the presence of the dihydrate form of ferrous gluconate, the spermiostatic effect was immediate. At lower concentrations of albumin and dextran, the spermiostatic effects were not significant. However, increasing the concentration of albumin to 10% decreased sperm motility almost close to that of 12.5 mM concentration of ferrous gluconate. In 20% albumin and 40% albumin almost 97% of the sperm were completely immobilized. In a 1.25% solution of dextran almost 95% of the sperm were immobilized.

There are concerns that initial releases of the active ingredients can be too high. Accordingly, a preferred embodiment of the invention includes a sheath over a portion of the device. This can limit the initial release rate and provide effective release for the duration of use, up to 31 days or longer.

Preferred embodiments of the invention are initially loaded with at least the following amounts of the following active agents as a weight percentage of the core matrix: at least 5%, preferably at least 10%, more preferably about 12.5% ferrous gluconate, at least 5%, preferably at least 10%, more preferably about 12.5% ascorbic acid and at least 5%, preferably at least 8%, more preferably about 10% polyglycolic acid.

Preferred embodiments of the invention include a core matrix, initially loaded as follows, with percentages by weight:
5% to 20%, preferably 10% to 15%, most preferably 12% to 13% Ferrous Gluconate
5% to 20%, preferably 10% to 15%, most preferably 12% to 13% Ascorbic Acid
5% to 20%, preferably 5% to 15%, most preferably 8% to 12% Sodium Dihydrogen Citrate
5% to 20%, preferably 5% to 15%, most preferably 8% to 12% Purasorb PG S polyglycolic acid (PGA)

To remain effective, the device should be releasing at least about 0.75, preferably about 1.0, more preferably at least 1.5 mg/day ferrous gluconate, and about at least 0.5, preferably at least 0.75, more preferably at least 1.0 mg/day ascorbic acid, as measured by storing the device in a 0.5% oxalic acid in water solution.

One preferred embodiment of the invention comprises a matrix of about 32-38% vinyl acetate EVA, combined with any of all of the following: about 10-15%, preferably 12-13% ferrous glutonate; about 10-15%, preferably 12-13% ascorbic acid; about 8-12%, preferably about 10% sodium dihydrogen citrate; about 8-12%, preferably about 10% polyglycolic acid.

The device should be constructed to provide continuous release of active agents. For example, in one embodiment of the invention, the device is adapted to release, continuously for at least 28 days, about 1.0, preferably about 1.5 mg/day ferrous gluconate and/or about 0.8, preferably about 1.0 mg/day ascorbic acid.

Ferrous gluconate is not toxic in appropriate amounts, can be biocompatible, and can be used as a nutritional iron supplement. Iron promotes lipid peroxidation. Lipid peroxidation is a type of cellular damage involving the formation of oxygen free radicals, such as super-oxide anion (Hong et al., “Effect of Lipid Peroxidation on Beating Frequency of Human Sperm Tail,” Andrologia 26:61-65 (1993); Aitken et al., “Relationship Between Iron-Catalyzed Lipid Peroxidation Potential and Human Sperm Function,” J. Reproduction and Fertility 98:257-265 (1993); and Calamer et al., “Effect of Lipid Peroxidation Upon Human Spermatic Adenosinetriphosphate (ATP). Relationship With Motility, Velocity and Linearity of the Spermatozoa,” Andrologia 21(1):48-49 (1988), which are hereby incorporated by reference in their entirety). Radicals are extremely unstable and unfavorable to the lipid bilayer of a cell resulting in cell damage. The lipid peroxidation process, as shown below, is initiated in human spermatozoa when intracellular production of reactive oxygen species overwhelms the antioxidant defense system, namely, superoxide dismutase (SOD), used by the cell.

Human spermatozoa are enriched with unsaturated fatty acids and fatty acids are particularly susceptible to lipid peroxidation. Sperm are thus predisposed to peroxidative damage. This reaction occurs when lipid peroxides in the bilayer of sperm tails are exposed to ferrous ion resulting in the propagation of lipid peroxidation, which leads to a continuous formation and decomposition of lipid peroxides. Eventually, this causes structural damage, a decline in metabolic activity, and spermiostatic effects in sperm cells. Ferrous gluconate targets sperm tail and can cause lipid peroxidation.

At the commencement of the menstrual cycle, cervical mucus has a tight honey-comb cellular structure with a channel diameter of 2-6 mμ, which forms an impenetrable barrier to sperm. At midcycle, the channel diameter is 30-35 mμ in order to allow the sperm to pass. At the luteal phase, the cellular structure again contracts to 2-6 mμ and the mucus becomes more viscous (WHO Laboratory Manual for the Examination of Human Semen and Sperm-cervical Mucus Interaction, Ch. 5:51-59 (1999), which is hereby incorporated by reference in its entirety). L-ascorbic acid is an antioxidant, transfers electrons, and acts as a reducing agent for disulfide (—S—S—) bonds of mucopolysaccharides of glycoproteins forming the cervical mucus, thus helping change the mucus from open cellular structure found at midcycle of the menstrual period to the closed cellular structure to form an impenetrable barrier for sperm.

It is evident that overall viscosity of the mucus increases in direct relationship with increasing concentrations of L-ascorbic acid from 0.310% to a range of 1-2.5%. The daily eluates for twelve consecutive days of a delivery device matrix containing L-ascorbic acid also increased the viscosity of the cervical mucus equivalent to that of normal follicular and luteal phases.

Devices in accordance with the invention are constructed so that the pH reducing agent, such as the ascorbic acid and the sperm immobilization agent, such a ferrous gluconate or ferrous sulfate, are released in a consistent manner. Preferably for 28-31 days or more, effective to contraceptively reduce sperm mobility and/or contraceptively increase cervical mucus impedance to sperm mobility. The device should include a releasable pH limiting agent to buffer (maintain) a pH in the vaginal cavity at a spermiostatic level. The device with the composition disposed therein should be constructed and formed with materials effective to promote controlled and continued sustained release of a contraceptively effective amount of the composition for at least 28-31 days or longer.

One preferred polymer for use in forming a device in accordance with the invention is ethylene-vinyl acetate (EVA), also known as poly (ethylene-vinyl acetate) (PEVA). EVA is the copolymer of ethylene and vinyl acetate (VA). The weight percent vinyl acetate can vary from 10 to 40%, with the remainder being ethylene. Therefore, commonly available EVA 28 refers to the copolymer having 28% vinyl acetate. Commonly available EVA 40 refers to the copolymer having 40% vinyl acetate.

It was determined that EVA 28 could not store sufficient therapeutic agents in an optimally sized device to promote adequate sustained therapeutic release. For example, it was determined that too much ferrous gluconate would be released from a device made from EVA 28 over too short a period of time to be suitable for a device designed to last 4 weeks. Accordingly, it was determined that a device in accordance with the invention is optimally a copolymer of ethylene and vinyl acetate, having more than 30% VA, preferably more than 32% VA. About 35% VA is preferred.

It was also determined that a device in accordance with the invention should have certain mechanical properties for proper handling and maintenance in the vagina for 4 weeks or longer. It was determined that EVA 40 was too soft for optimal construction of the device. Accordingly, it was determined that a device in accordance with the invention is optimally a copolymer of ethylene and vinyl acetate, having less than 40% VA, preferably less than about 38% VA. Preferred devices in accordance with the invention should have about 32-38% VA, preferably about 34-36% VA, most preferably about 35% VA. The remainder is ethylene.

It was also been determined that by applying a sheath, as discussed more fully below, around the EVA device, the rate, in particular, the initial rate at which the active ingredients are released from the device can be slowed. Therefore, the area confined by the sheath can act as a reservoir of active ingredients, which can migrate over time to the unsheathed regions. Accordingly, by sheathing a section of the device, less of the initial loading will be released in the first week or two, and therefore, more will remain stored in the device. Hence, it is believed that the sheathed area can act as a reservoir of active ingredients, which then migrate to and are released from the unsheathed regions. As used herein, % sheath will refer to surface area of the device covered with the sheath and not wt %. Preferred sheathing is over about 10%, preferably about 10% to 50%, more preferably about 15% to 35%, most preferably about 25%.

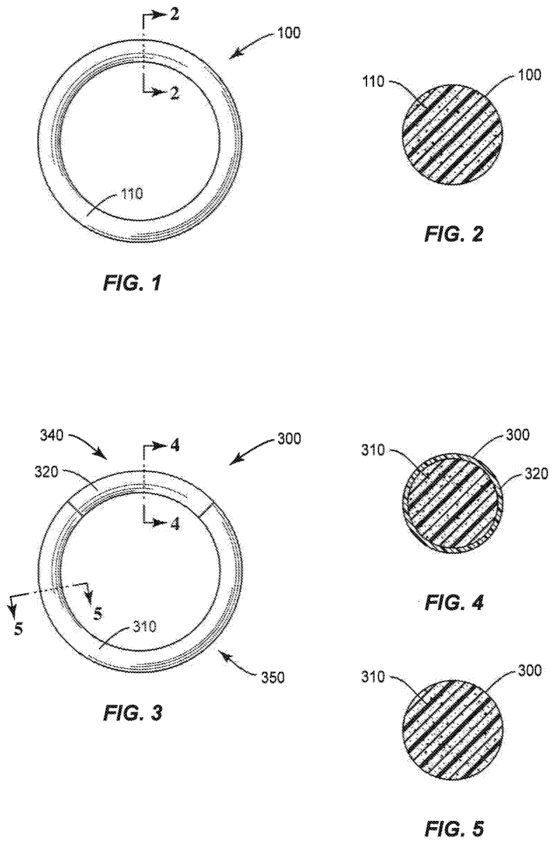

A perspective view of an unsheathed vaginal ring 100 is shown in FIG. 1. The cross-section of vaginal ring 100, taken along a line 22 is shown in FIG. 2. Vaginal ring 100 is constructed to be drug-loaded essentially uniformly across cross-section 110. Thus, ring 100 presents a monolithic matrix.

A perspective view of a sheathed vaginal ring 300 is shown in FIG. 3. A cross-sectional view of a sheathed section 340 of vaginal ring 300, taken along line 4-4, is shown in FIG. 4. A cross-section of an unsheathed section 350 of vaginal ring 300, taken along line 55, is shown in FIG. 5. Ring 300 includes a drug-loaded monolithic matrix 310. Sheathed section 340 includes a sheath 320 over drug-loaded monolithic core section 310. Sheathed section 340 represents about 25% of the surface area of ring 300. Sheath 320 should be resistant to transmission of the active ingredients and can prevent and/or restrict the elution of drugs from core 310. Thus, as drugs are eluted from unsheathed section 350, drugs in sheathed section 340 can provide a reservoir to recharge unsheathed section 350, as drugs are released from section 350. By adjusting the size of sheathed section 340, the amount of restriction can be controlled, to reduce the initial release of drugs from unsheathed section 350.

Preferred materials for the sheath include: EVA, preferably EVA 28. The sheath preferably has a thickness of about 80 to 120 μm, preferably about 100 μm.

The following examples of devices and agents to be loaded into the devices are presented for purposes of illustration and should not be construed as limiting the scope of the invention.

### Example 1

One preferred Target Product Profile (TPP) is as follows:

Matrix composition, which can be partially covered with a sheath:

12.5 5 Ferrous Gluconate (FG)

12.5% Ascorbic Acid (AA)

10% Sodium dihydrogen citrate (MSC)

10% Purasorb PG S (PGA)\

55% EVA35

Ethylene Vinyl acetate copolymer, 35% vinyl acetate content (EVA35), manufactured from a blend of EVA28 and EVA40 at a ratio of 41.7:58.3% respectively. All percentages are by weight.

Target In-vitro elution (IVE):

FG: minimum 1.5 mg/day

AA: minimum 1.0 mg/day

Device Design:

Matrix (Monolithic) IVR

25%-Sheathed IVR

Matrix (Monolithic) IVR

Composition

Mass ratio of all components in the Core Matrix IVR

12.5% Ferrous Gluconate

12.5% Ascorbic Acid

10% Sodium Dihydrogen Citrate

10% Purasorb PG S polyglycolic acid (PGA)

55% EVA35

To form a device in accordance with a preferred embodiment of the invention, the EVA 28 and EVA 40 was blended (41.7% EVA 28 and 58.3% EVA 40) to yield EVA 35 with a Cryo-mill mixing and hotmelt extrusion. The material was then palletized, melted and subjected to low temperature extrusion (100° C.) to form the device substrate. The EVA 35 substrate material was then mixed with the PGA (84.6% EVA 35 and 15.4% PGA) by cryo-milling, high temperature mixing (215° C.), hotmelt extrusion, palletization and then high temperature extrusion.

The ferrous gluconate, ascorbic acid and MSC were combined by cryo-milling, mixing, hotmelt extrusion, palletization and low temperature extrusion. The milled EVA 35/PGA powder was dry blended with the FG, AA and MSC to produce a mixture of 12.5% FG, 12.5% AA, 10% MSC and 65% PGA-loaded EVA 35. The loaded matrix stand was then formed into pellets by low temperature (100° C.) extrusion. 5.5 mm diameter strands were formed at 87° C. and cut to 157 mm long strands and the ring-shape was formed by thermal welding. Further shaping to form the ring into a circle was performed at 65° C., as needed to form rings.

The mixing of the EVA 35 and PGA was performed with high temperature extrusion, to ensure that the PGA was adequately melted and mixed into the EVA 35. The EVA 35/PGA mixture was combined with the FG, AA and MSC at low temperature to prevent any thermal degradation of the API's.

The ring comprised a matrix section, 75% unsheathed core IVR and a core-sheath section, 25% IVR. The core-sheath section was covered with a 100 μm sheath of EVA 28.

Matrix Section Composition

Mass ratio of all components in the Matrix section

12.5% Ferrous Gluconate

12.5% Ascorbic Acid

10% Sodium Dihydrogen Citrate

10% Purasorb PG S (PGA)

55% EVA35

Core-sheath Section Composition

Mass ratio of all components in the core-sheath section

11.61% Ferrous Gluconate

11.61% Ascorbic Acid

9.29% Sodium Dihydrogen Citrate

9.29% Purasorb PG S

51.07% EVA35

7.14% EVA28 (sheath material)

To form a section covered with a sheath, extrusion of the EVA 35/PGA mixture that had been combined with the FG, AA and MSC is co-extruded through a dual layer cross head. A length of the core-sheath strand is cut to 39 mm and the matrix strand was cut to a length of 118 mm. The matrix strand is then joined to the core-sheath strand with thermal welding and then formed into a ring. To form the sheath, EVA 28 was melted and extruded through the sheath section of the dual layer cross head. The core-sheath extruded eight was formed into a cylindrical strand of 5.5 mm by extrusion at a temperature of 87° C.

### Example 2

The use of a sheathed matrix has shown to slow the initial release of active agents and thereby, preserve more of the active agents for long term release. For example, when a ring was prepared as in Example 1, but with EVA35 and 35%, 25%, 15% and 0% sheathing, the initial release of ferrous gluconate was reduced, dramatically, thereby preserving more of the ferrous gluconate within the ring, as seen in Table 1.

TABLE 1
Daily Release From EVA35 IVR

| Day | % Sheath and Daily Ferrous Gluconate Release (mg) | | | |
|---|---|---|---|---|
| 1 | 0% = 23.2 mg | 15% = 15.9 mg | 25% = 13.7 mg | 35% = 13.2 mg |
| 2 | 0% = 11.6 mg | 15% = 8.4 mg | 25% = 7.0 mg | 35% = 6.9 mg |
| 3 | 0% = 8.4 mg | 15% = 6.8 mg | 25% = 6.8 mg | 35% = 4.9 mg |
| 4 | 0% = 7.5 mg | 15% = 5.8 mg | 25% = 5.2 mg | 35% = 5.1 mg |

The release of ascorbic acid was also shown to be restricted by the use of sheathing, as shown in Table 2.

TABLE 2
Daily Release From EVA 35 IVR

| Day | % Sheath and Daily Ascorbic Acid Release (mg) | | | |
|---|---|---|---|---|
| 1 | 0% = 27.1 mg | 15% = 24.1 mg | 25% = 25.5 mg | 35% = 21.8 mg |
| 2 | 0% = 16.3 mg | 15% = 11.8 mg | 25% = 11.3 mg | 35% = 10.3 mg |
| 3 | 0% = 12.9 mg | 15% = 8.2 mg | 25% = 8.0 mg | 35% = 7.6 mg |
| 4 | 0% = 10.1 mg | 15% = 6.7 mg | 25% = 7.0 mg | 35% = 6.9 mg |

It will thus be seen that the objects set forth above, among those made apparent from the preceding description, are efficiently attained, and, since certain changes may be made in carrying out the above method and in the article of manufacture set forth, without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of the scope of the invention which, as a matter of language, might be said to fall therebetween.

Particularly it is to be understood that in said claims, ingredients or compounds recited in the singular are intended to include compatible mixtures of such ingredients wherever the sense permits.

What is claimed is:

1. A biocompatible intravaginal device, adapted for the delivery of therapeutic agents, comprising:
an ethylene-vinyl acetate (EVA) copolymer substrate adapted to be inserted into and remain in the vagina for over 7 days, formed of at least 30 wt % vinyl acetate; and
the substrate releasably loaded with active agents and containing a contraceptively effective amount of at least one contraceptive active agent selected from the group consisting of calcium chloride, magnesium chloride, ferrous sulfate, copper sulfate, or ferrous gluconate in addition to at least one pH reducing active agent selected from ampholines or ascorbic acid.

2. The intravaginal device of claim 1, wherein the device is constructed and adapted to be inserted in a vagina of a fertile woman and release a contraceptively effective amount of the contraceptive agent or pH reducing agent to act as a contraceptive, continuously, for at least 28 days.

3. The intravaginal device of claim 2, wherein the EVA of the substrate comprises 32 to 38% vinyl acetate.

4. The intravaginal device of claim 2, wherein the substrate has a ring shape around a central region with at least one opening in the central region and substantially lacks hormonal ingredients.

5. The intravaginal device of claim 2, wherein the substrate is loaded and adapted to release, continuously, for at least 28 days, as measured in a 0.5% oxalic acid in water solution, at least about 1.0 mg/day ferrous gluconate.

6. The intravaginal device of claim 2, wherein the substrate is loaded and adapted to release, continuously, for at least 28 days, as measured in a 0.5% oxalic acid in water solution, at least about 0.5 mg/day ascorbic acid.

7. The intravaginal device of claim 2, wherein the substrate is loaded and adapted to release, continuously, for at least 28 days, as measured in a 0.5% oxalic acid in water solution, at least about 1.0 mg/day ferrous gluconate and at least about 0.5 mg/day ascorbic acid.

8. The intravaginal device of claim 1, wherein a portion of the substrate is covered with a sheath that restricts the release of the active agent from the sheathed portion of the device.

9. The intravaginal device of claim 1, wherein the substrate is in the shape of a ring and a portion of the substrate is covered with a sheath that restricts the release of the active agent from the sheathed portion of the device.

10. The intravaginal device of claim 8, wherein the substrate has an elongated portion having a circular cross section having a diameter of 4 to 7 mm and the sheath encircles a portion of the elongated portion.

11. The intravaginal device of claim 10, wherein the sheath has a thickness of 80 to 120 μm.

12. The intravaginal device of claim 11, wherein the sheathed portion of the substrate is formed by co-extruding the sheath over the substrate portion having the active agents therein.

13. The intravaginal device of claim 12, wherein the substrate is formed as connected portions of unsheathed and sheathed portions.

14. The intravaginal device of claim 1, wherein the contraceptively active agents comprise about 10-15% ferrous gluconate or ferrous sulfate and about 10-15% ascorbic acid.

15. The intravaginal device of claim 1, wherein the contraceptively active agents consist essentially of about 10-15% ferrous gluconate or ferrous sulfate, about 10-15% ascorbic acid, about 8-12% sodium dihydrogen citrate, and about 8-12% polyglycolic acid.

16. The intravaginal device of claim 1, wherein the contraceptively active agents comprise about 12-13% ferrous gluconate, about 12-13% ascorbic acid and pH buffers.

17. The intravaginal device of claim 1, wherein the contraceptively active agents consist essentially of about 12.5% ferrous gluconate, about 12.5% ascorbic acid, about 10% sodium dihydrogen citrate, and about 10% polyglycolic acid.

18. The intravaginal device of claim 15, and comprising anti-bacterial or anti-viral agents.

19. The intravaginal device of claim 15, and comprising clindamycin, metronidazole, clindamycin, tenofovir, emtricitabine, miconazole, clotrimazole, butoconazole, or tioconazole.

20. A method of forming at least a portion of an insertable medical device, comprising:
blending polymer material with active agents to form a substrate loaded with the active agents therein;
co-extruding a portion of the loaded substrate with a polymer sheath, wherein the sheath covers the loaded substrate to form extruded strands of a sheathed loaded substrate;
extruding another portion of the loaded substrate to form unsheathed loaded substrate strands;
combining the sheathed substrate strands with the unsheathed substrate strands to form a partially sheathed loaded substrate.

21. The method of claim 20, wherein the active agents are contraceptive materials and the device functions as a contraceptive.

22. The method of claim 16, wherein the sheathed substrate strands are thermally welded to the unsheathed substrate strands to form a ring shape.
